(19) United States
(12) Patent Application Publication
(10) Pub. No.: US 2022/0218983 A1
(43) Pub. Date: Jul. 14, 2022

(54) Conductive Member For Use In Radiofrequency Ablation

(71) Applicant: Ablatus Therapeutics Limited, Cambridge (GB)

(72) Inventors: David Graham Brooks, Withersfield Suffolk (GB); Christopher Paul Wickham French, Bedford (GB); Daniel Peterson Godfrey, Cambridge (GB); Scott Virgo, Swaffham Bulbeck (GB); David Seymour Warwick, Cambridge (GB)

(21) Appl. No.: 17/613,853

(22) PCT Filed: May 22, 2020
(86) PCT No.: PCT/IB2020/054881

§ 371 Date: Nov. 23, 2021

(30) Foreign Application Priority Data
May 23, 2019 (GB) 1907269.3

(51) Int. Cl. A61N 1/04; A61B 18/12; A61B 18/14; A61B 18/16

(52) U.S. Cl. CPC A61N 1/0492; A61B 18/1206; A61B 2018/00071; A61B 18/16; A61N 1/048; A61B 18/1477

(57) ABSTRACT

A conductive member such as conductive pad (1) for use in radiofrequency ablation, the conductive member being flexible so as to conform to a subject's skin, the conductive member comprising a conductive skin contact layer (5) arranged for contact with the subject's skin and a conductive layer (4) over the conductive skin contact layer, in which the conductive skin contact layer (5) and the conductive layer (4) are both conductive to DC electrical signals.

### CROSS-REFERENCE TO RELATED APPLICATION(S)

This application is a 371 application of International Application No. PCT/IB2020/054881, filed on May 22, 2020, which claims priority to U.K. Patent Application No. 1907269.3, filed on May 23, 2019, the entire disclosures of all of which are hereby incorporated by reference.

### TECHNICAL FIELD

This disclosure relates to a conductive member for use in radiofrequency ablation, a radiofrequency ablation system and a method of using such a system.

### BACKGROUND

Radiofrequency ablation as a method of ablating tissues within a subject is known; a radiofrequency electric signal is applied to the tissues to be ablated using a (typically) pointed electrode, with return current typically being collected through a conductive member such as a pad applied to the subject's skin. For the subject's comfort and to avoid injury to the subject, it is desirable to keep the current per unit area passing through the conductive member as small as possible.

We are aware of the PCT patent application published as WO2006/082413, which discloses the use of such a radiofrequency ablation system with a DC offset applied to the radiofrequency signal to reduce the desiccating effect of radiofrequency ablation on the tissue to be ablated. That discusses using a conductive pad together with a separately applied (and “conventional”) conductive gel. This can be referred to as “bimodal electric tissue ablation”.

### SUMMARY

In accordance with a first aspect of the disclosure, we provide a conductive member for use in radiofrequency ablation, the conductive member being flexible so as to conform to a subject's skin, the conductive member comprising a conductive skin contact layer arranged for contact with the subject's skin and a conductive layer over the conductive skin contact layer, in which the conductive skin contact layer and the conductive layer are both conductive to DC electrical signals.

As such, by providing a conductive skin contact layer as part of a conductive member, this allows for improved contact with a subject's skin. Furthermore, making the conductive skin contact layer and the conductive layer conductive to DC signals is advantageous when using bimodal electric tissue ablation, where there is a DC component to the excitation signal. We have appreciated that prior art electrodes with prior art conductive gels are poor conductors of DC signals, and that transmission of the DC component is advantageous in bimodal electric tissue ablation. By reducing the resistance to DC signals, lower voltages are required to achieve a target DC current, which reduces the potential risks (e.g. unintended electro-muscular stimulation) to the subject.

The conductive member may be a conductive pad, which may be arranged to adhere, typically by means of the conductive skin contact layer, to the subject's skin. Alternatively, the conductive member may not be adhesive to a user's skin; in such a case the conductive member may comprise attachment means, such as a resilient member, by means of which the conductive member (typically the conductive skin contact layer) can be held in use against the subject's skin.

In one embodiment, the conductive member may be arranged so as to be wearable. Typically, the conductive member would comprise a compression member which is wearable on a part of the subject (typically an arm or a leg) and which is placed in tension by being worn. The tension in the compression member may act to hold the conductive member, and in particular the conductive skin contact layer, against the subject's skin.

Typically, the conductive member would comprise an input for an electrical signal, coupled to the conductive layer. The input may comprise a conductive projection from the conductive layer.

The conductive skin contact layer may have a volume resistivity at a frequency less than 5 Hz, or at zero frequency (i.e. DC) of a maximum of 2500 ohm cm, or 2000 ohm cm, or 1500 ohm cm, or 1000 ohm cm.

The conductive skin contact layer may comprise a gel layer, which may comprise a hydrogel which is conductive to DC signals. The conductive skin contact layer may be between 0.5 and 1 mm thick.

The conductive layer will typically comprise a conductive plastic material, such as carbon-loaded polymer mix. In one embodiment, the conductive layer will comprise a carbon-loaded polyethylene film. The conductive layer may have a surface resistivity of at most 300, or 250, Ohms per square. The conductive layer may be between 0.1 and 0.5 mm thick. We have found that using such a conductive plastic material avoids any reaction between the conductive skin contact layer and a metallic conductive layer, both during storage of the conductive member (where in particular a hydrogel could corrode a metal electrode) and during use (where gas can evolve at the interface between the conductive skin contact layer and a metallic conductive layer).

Additionally or alternatively, the conductive layer may comprise any of conductive fabrics, metal loaded substrates, sheet metal foils, conductive meshes, metallized fabric, or conductive silicones.

The conductive member may also comprise a removable release layer on the conductive skin contact layer, to protect and hold captive the conductive skin contact layer until it is applied to the subject's skin. Typically, the release layer will comprise silicone-coated polymer material, such as silicone-coated polyethylene terephthalate (PET).

The conductive member may also comprise a foam layer over the conductive layer, typically on the side of the conductive layer opposite to the conductive skin contact layer. This can provide support to the other layers and also provide insulation to protect medical operators from any electrical signals. The foam layer may comprise a medical foam, such as a closed cell polyethylene foam. The foam layer may be attached to the conductive layer by means of an intervening adhesive layer. The adhesive layer may comprise two layers of a conductive adhesive over a non-woven fabric core. The use of a fabric core can help avoid corrosion of a metallic substrate by the high salt content in the hydrogel where a hydrogel is used as the gel layer.

In accordance with a second aspect of the disclosure, there is provided a radiofrequency ablation system, comprising a conductive member in accordance with the first aspect of the disclosure, an electrode and a radiofrequency source arranged to generate a signal with a radiofrequency component, and coupled to the electrode and the conductive member to apply the signal between the electrode and the conductive member.

Typically, the signal will have a DC component; as such, the radiofrequency ablation system may be for use with bimodal electric tissue ablation.

The electrode may comprise a pointed needle, typically metal, in electrical communication with the radiofrequency source.

The radiofrequency component will typically have a frequency in the range of 300 to 600 kHz (typically 400 to 500 kHz). The signal will typically have a power of between 20 to 200 watts.

The DC component will typically have a voltage of a maximum of 40 volts, typically between 0 and 25 volts.

In accordance with a third aspect of the disclosure, there is provided a method of ablating a subject's tissue using the radiofrequency ablation system of the second aspect of the disclosure, the method comprising applying the conductive member to the subject's skin, positioning the electrode adjacent to the tissue to be ablated and passing the signal from the electrode through the tissue to be ablated, the signal returning to the conducting pad through the subject.

The signal may be applied to the tissue for at least 1 minute, 5 minutes, 10 minutes, 15 minutes or 20 minutes.

There now follows, by way of example, description of an embodiment of the disclosure, described with reference to the accompanying drawings, in which:

### BRIEF DESCRIPTION OF DRAWINGS

FIG. 1 shows an exploded view of a conductive member in accordance with an embodiment of the disclosure;

FIG. 2 shows a plan view of the conductive member of FIG. 1, showing the internal arrangement of the components forming the conductive member;

FIG. 3 shows a side elevation of the conductive member of FIG. 1;

FIG. 4 shows an enlargement of area A of FIG. 3;

FIG. 5 shows a plan view of the foam layer of the conductive member of FIG. 1;

FIG. 6 shows a plan view of the adhesive layer of the conductive member of FIG. 1;

FIG. 7 shows a plan view of the conductive layer of the conductive member of FIG. 1;

FIG. 8 shows a plan view of the gel layer of the conductive member of FIG. 1;

FIG. 9 shows a plan view of the release layer of the conductive member of FIG. 1;

FIG. 10 shows schematically a radiofrequency ablation system in accordance with the present disclosure, using the conductive member of FIG. 1.

### DETAILED DESCRIPTION

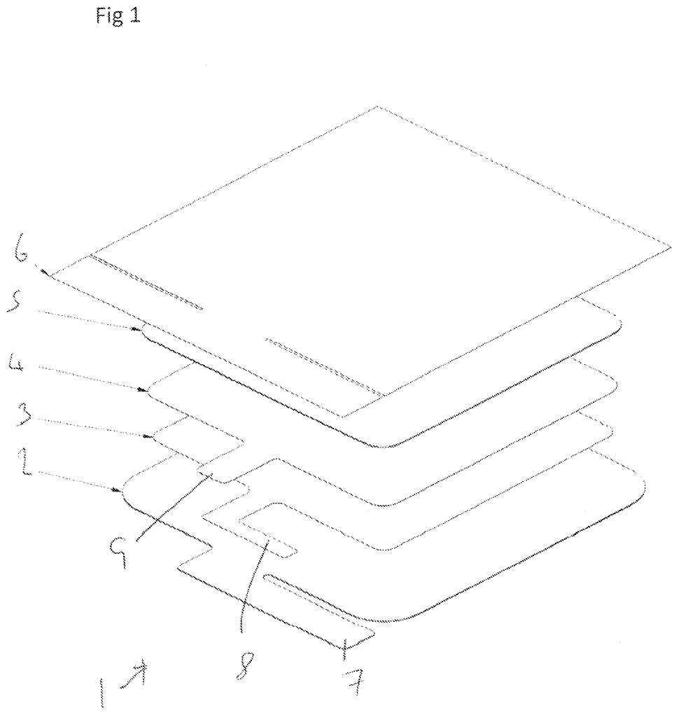

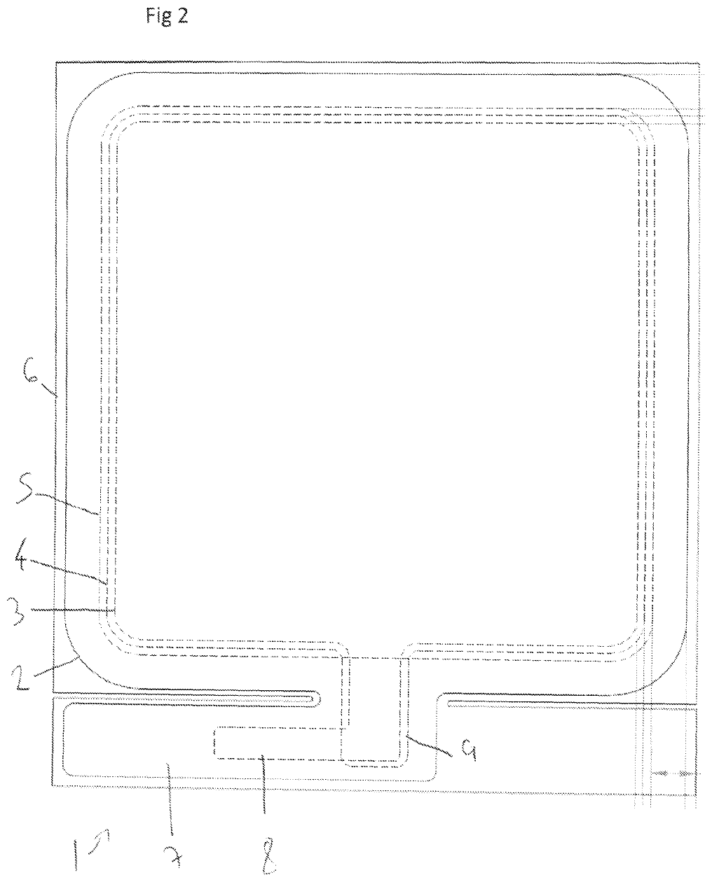

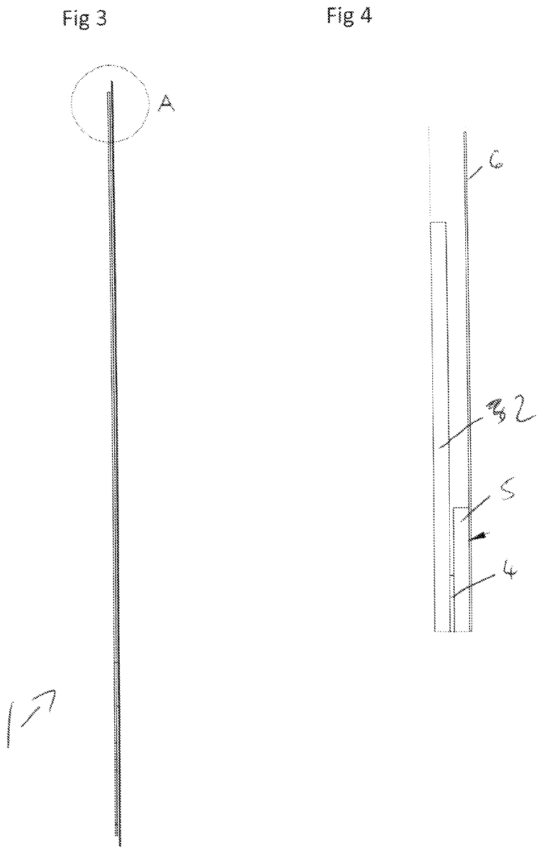

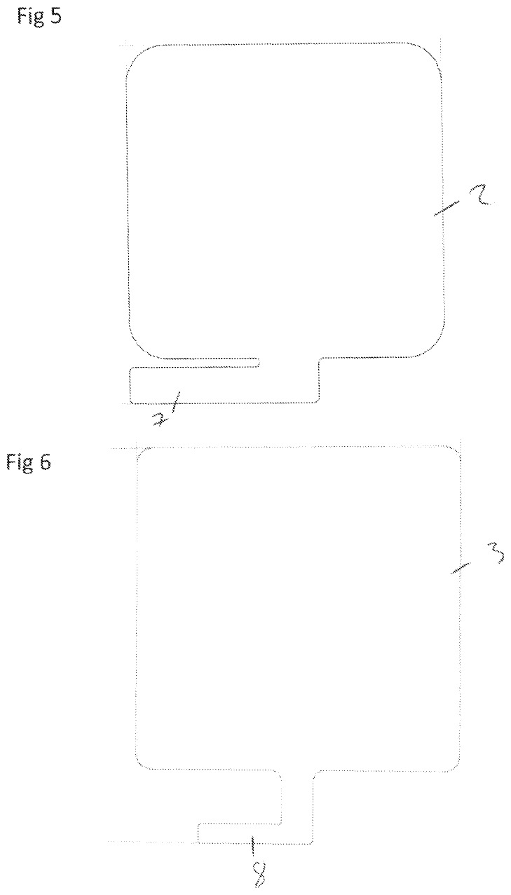

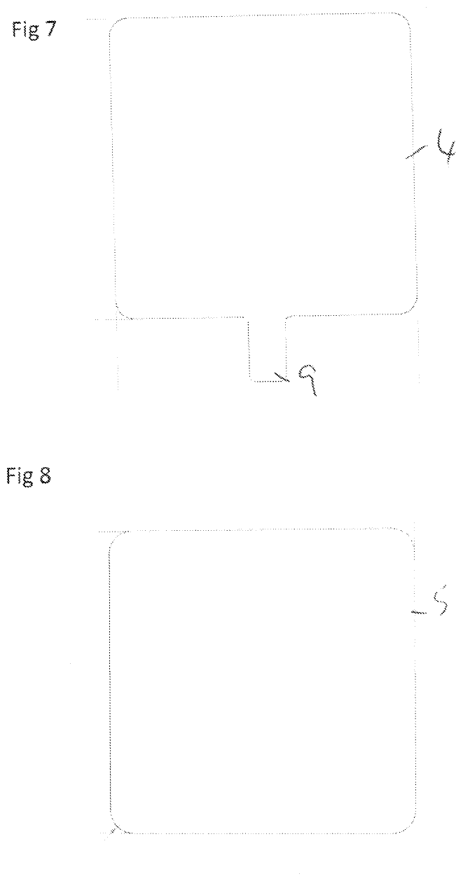

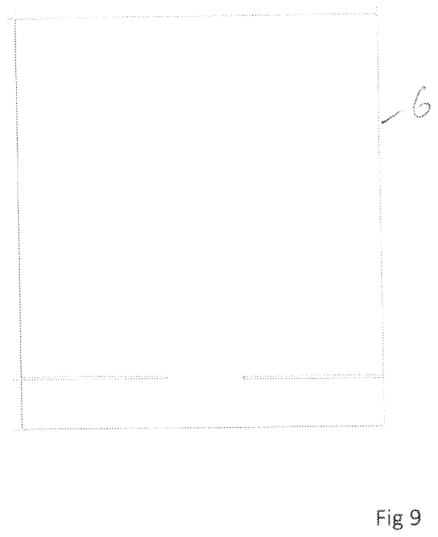

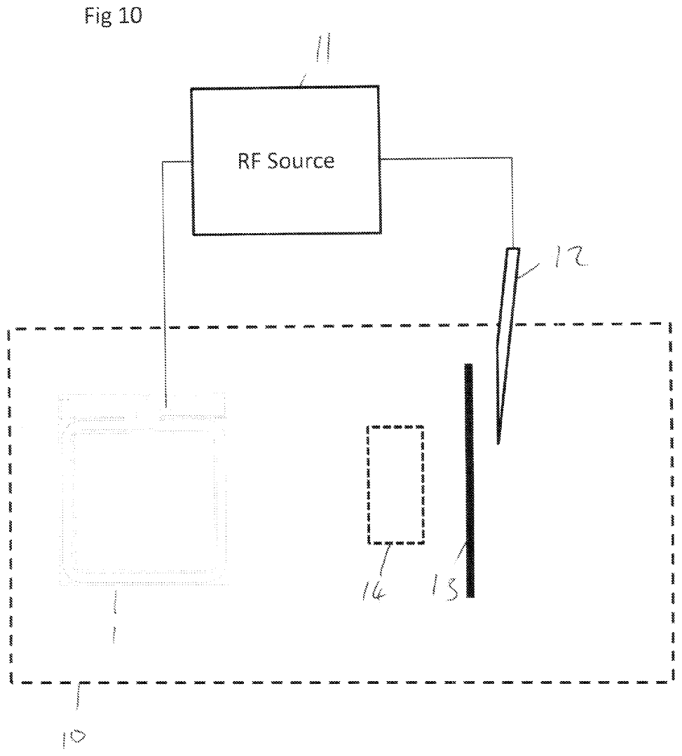

A conductive member of the form of a conductive pad 1 for use in radiofrequency ablation is shown in FIGS. 1 to 9 of the accompanying drawings; it is shown as part of a radiofrequency ablation system in FIG. 10 of the accompanying drawings. The conductive pad 1 comprises a number of layers built into a flexible pad.

Taking the layers in turn, from the bottom of FIG. 1 upwards:
a foam layer 2;
an adhesive layer 3;
a conductive layer 4;
a conductive skin contact layer, of the form of a gel layer 5; and
a release layer 6.

The foam layer 2 provides structure to the conductive pad 1. It comprises a single-sided medical closed cell polyethylene foam, such as product 9776 from 3M Medical Specialities of St Paul, Minn., USA. The foam layer is 0.7 mm thick. It is shaped as a rounded rectangle, with a tail portion 7 extending parallel to one side of the rectangle.

On the foam layer is provided the adhesive layer 3. This is provided as a layer of conductive non-woven fabric with conductive adhesive on both sides, such as the tape sold as HB350 from Hi-Bond Tapes Ltd of Corby, United Kingdom. Again, this is formed of rounded rectangular body, smaller than the foam layer 2, with a tail portion 8 that fits within tail portion 7 of the foam layer 2. The area around tail portion 7 connection needs to be fully covered by an insulating material (typically the backing layer) to avoid any risk of short circuit to the patient or operator. The adhesive layer is 0.1 mm thick.

On top of the adhesive layer is the conductive layer 4. This comprises a polyethylene film loaded with carbon, such as the film available as LINQSTAT XVCF from Caplinq, Heemskirk, Netherlands. It is again of the form of a rounded rectangle, with a tab 9 for the connection to a signal generator (discussed below). The conductive layer 4 is larger than the adhesive layer, but smaller than the foam layer 2. The conductive layer 4 is 0.2 mm thick.

On top of the conductive layer 4 is the gel layer 5. This comprises a hydrogel, such as that sold as AG625 from Axelgaard Manufacturing Co, Ltd of Fallbrook, Calif., USA. This allows the conductive pad 1 to conform to a subject's skin, and provides some adhesion to the subject's skin. The gel layer is provided as a rounded rectangle, larger than the conductive layer 4 but smaller than the foam layer 2. The gel layer is 0.7 mm thick.

The conductive layer 4 and the gel layer 5 are together conductive to a range of frequencies from DC (zero frequency) up to at least 1 MHz.

On top of the gel layer 5 is provided a release layer 6 of the form of silicone-coated polyethylene terephthalate. This protects and retains the gel layer 5 until it is ready to be used. The silicone coating allows for the easy removal of the release layer 6.

The use of the conductive pad 1 is shown in FIG. 10 of the accompanying drawings. The conductive pad 1 is used as part of a radiofrequency ablation system along with a radiofrequency source 11 and a needle electrode 12.

The conductive pad 1 (with the release layer 6 removed) is applied to a subject's skin 10, so that the gel layer 5 adheres to the subject's skin 10. It is connected to the radiofrequency source 11, as is the needle electrode 12. The radiofrequency source 11 is used to create a signal applied as a voltage between the needle electrode 12 and the conductive pad 1. The signal has a radiofrequency component at around 460 kHz supplying between 20 and 200 W. It also has a DC component of between 0 and 40 volts (with the conductive pad 1 as the anode), which will be transmitted by the conductive layer 4 and the gel layer 5 as they transmit DC signals.

Typically the resistance measured through the patient as a result of using the hydrogel will be less than 500 ohm the resistance is largely driven by the impedance of the stratum corneum which can be greater 1 megaohm per cm2. A sufficient area of hydrogel is used to overcome this.

As such, the needle electrode 12 can be introduced into an incision 13 in the user's skin 10 and used to ablate a tissue 14 of interest. The DC component will reduce the dehydrating effect of the tissue ablation.

1. A conductive member for use in radiofrequency ablation, the conductive member being flexible so as to conform to a subject's skin, the conductive member comprising a conductive skin contact layer arranged for contact with the subject's skin and a conductive layer over the conductive skin contact layer, in which the conductive skin contact layer and the conductive layer are both conductive to DC electrical signals.

2. The conductive member of claim 1, being a conductive pad.

3. The conductive member of claim 1, which is arranged to adhere, typically by means of the conductive skin contact layer, to the subject's skin.

4. The conductive member of claim 1, in which the conductive member is not adhesive to a user's skin.

5. The conductive member of claim 1, comprising attachment means by means of which the conductive member (typically the conductive skin contact layer) can be held in use against the subject's skin.

6. The conductive member of claim 1, arranged so as to be wearable, and comprising a compression member which is wearable on a part of the subject and which is placed in tension by being worn, tension in the compression member acting to hold the conductive member, and in particular the conductive skin contact layer, against the subject's skin.

7. The conductive member of claim 1, in which the conductive skin contact layer has a volume resistivity at a frequency less than 5 Hz, or at zero frequency (i.e. DC) of a maximum of 2500 ohm cm, or 2000 ohm cm, or 1500 ohm cm, or 1000 ohm cm.

8. The conductive member of claim 1, in which the conductive skin contact layer comprises a gel layer.

9. The conductive member of claim 8, in which the gel layer comprises a hydrogel which is conductive to DC signals.

10. The conductive member of claim 1, in which the conductive layer comprises a conductive plastic material, such as carbon-loaded polymer mix.

11. The conductive member of claim 1, comprising a removable release layer on the conductive skin contact layer.

12. The conductive member of claim 1, comprising a foam layer over the conductive layer on the side of the conductive layer opposite to the conductive skin contact layer.

13. The conductive member of claim 12, in which the foam layer is attached to the conductive layer by means of an intervening adhesive layer.

14. The conductive member of claim 13, in which the adhesive layer comprises two layers of a conductive adhesive over a non-woven fabric core.

15. A radiofrequency ablation system, comprising a conductive member in accordance with claim 1, an electrode and a radiofrequency source arranged to generate a signal with a radiofrequency component, and coupled to the electrode and the conductive member to apply the signal between the electrode and the conductive member.

16. The system of claim 15, in which the signal has a DC component.

17. The system of claim 15, in which the electrode comprises a pointed needle.

18. A method of ablating a subject's tissue using the radiofrequency ablation system of claim 15, the method comprising applying the conductive member to the subject's skin, positioning the electrode adjacent to the tissue to be ablated and passing the signal from the electrode through the tissue to be ablated, the signal returning to the conducting pad through the subject.
